(19) United States
(12) Patent Application Publication
(10) Pub. No.: US 2022/0071542 A1
(43) Pub. Date: Mar. 10, 2022

(54) BIOLOGICAL INFORMATION MEASUREMENT APPARATUS

(71) Applicants: SONY GROUP CORPORATION, TOKYO (JP); SONY SEMICONDUCTOR SOLUTIONS CORPORATION, KANAGAWA (JP)

(72) Inventors: MAO KATSUHARA, TOKYO (JP); KAZUNARI YOSHIFUJI, KANAGAWA (JP); YUKI YAGISHITA, KANAGAWA (JP); TAKESHI OHKAWA, KANAGAWA (JP)

(21) Appl. No.: 17/309,951

(22) PCT Filed: Jan. 8, 2020
(86) PCT No.: PCT/JP2020/000238

§ 371 Date: Jul. 2, 2021

(30) Foreign Application Priority Data
Jan. 18, 2019 (JP) 2019-006749

(51) Int. Cl. A61B 5/304; A61B 5/00; A61B 5/25; A61B 5/053

(52) U.S. Cl. CPC A61B 5/304; A61B 5/053; A61B 5/25; A61B 5/7203

(57) ABSTRACT

A biological information measurement apparatus according to an embodiment of the present disclosure includes one or a plurality of measurement channels to be brought into contact with a biological body, and a reference channel to be brought into contact with the biological body. The biological information measurement apparatus further includes a differential circuit that generates a biological signal corresponding to a difference between a measurement signal obtained from the measurement channel and a reference signal obtained from the reference channel, and a switch mechanism that switches contact impedance between the biological body and each of the measurement channel and the reference channel.

### TECHNICAL FIELD

The present disclosure relates to a biological information measurement apparatus.

### BACKGROUND ART

Noises occurring as a result of an external electromagnetic wave being coupled to and mixed into a human body or a wiring line have become a major issue in an apparatus that measures a minute electric potential difference such as a brain wave. Among such alternate noises, a noise as a result of electrostatic induction with the human body is usually reduced by a differential circuit. However, when a difference occurs in contact impedances between the biological body and two electrodes coupled to the differential circuit, the alternate noise remains without being able to be removed by the differential circuit, which is an issue. To address this issue, various measures have heretofore been proposed (see, for example, PTL 1)

### CITATION LIST

### Patent Literature

PTL 1: Japanese Unexamined Patent Application Publication No. 2014-124438

### SUMMARY OF THE INVENTION

Incidentally, in an apparatus that measures biological information such as a brain wave, a contact status of an electrode may be changed due to body movement or insufficient attachment. When the contact status of the electrode is changed, the contact impedance is also changed accordingly, thus making it difficult to effectively remove the alternate noise included in the biological information. It is therefore desirable to provide a biological information measurement apparatus that makes it possible to effectively reduce an alternate noise included in biological information even under a circumstance where a contact status of an electrode may be changed.

A biological information measurement apparatus according to an embodiment of the present disclosure includes one or a plurality of measurement channels to be brought into contact with a biological body, and a reference channel to be brought into contact with the biological body. The biological information measurement apparatus further includes a differential circuit that generates a biological signal corresponding to a difference between a measurement signal obtained from the measurement channel and a reference signal obtained from the reference channel, and a switch mechanism that switches contact impedance between the biological body and each of the measurement channel and the reference channel.

In the biological information measurement apparatus according to an embodiment of the present disclosure, the differential circuit is provided that generates a biological signal corresponding to a difference between the measurement signal and the reference signal, and a switch mechanism is further provided that switches contact impedance between the biological body and each of the measurement channel and the reference channel. This makes it possible to adjust the contact impedance depending on the contact status of the channels.

### BRIEF DESCRIPTION OF THE DRAWINGS

FIG. 1 illustrates an example of a schematic configuration of a biological information measurement apparatus according to an embodiment of the present disclosure.

FIG. 2 illustrates an example of a circuit configuration of a measurement electrode module of FIG. 1.

FIG. 3A illustrates an example of a circuit configuration of a reference electrode module of FIG. 1.

FIG. 3B illustrates an example of the circuit configuration of the reference electrode module of FIG. 1.

FIG. 4 illustrates a cross-sectional configuration example of the measurement electrode module of FIG. 2.

FIG. 5 illustrates a cross-sectional configuration example of the reference electrode module of FIG. 3A.

FIG. 6 illustrates a perspective configuration example of the measurement electrode module and the reference electrode module of FIG. 1.

FIG. 7 illustrates a perspective configuration example of the measurement electrode module and the reference electrode module of FIG. 1.

FIG. 8 illustrates an example of procedures of impedance matching and biological signal acquisition in the biological information measurement apparatus of FIG. 1.

FIG. 9A illustrates an example of a biological signal at the time when impedance mismatching is large.

FIG. 9B illustrates an example of a biological signal at the time when the impedance mismatching is small.

FIG. 9C illustrates an example of a biological signal at the time when the impedance matching is obtained.

FIG. 10 illustrates a modification example of the circuit configuration of the measurement electrode module of FIG. 2.

FIG. 11 illustrates a modification example of the circuit configuration of the measurement electrode module of FIG. 2.

FIG. 12 illustrates an example of procedures of the impedance matching and the biological signal acquisition in the biological information measurement apparatus including any of the measurement electrode modules of FIGS. 10 and 11.

FIG. 13 illustrates a modification example of the circuit configuration of the measurement electrode module of FIG. 2.

FIG. 14 illustrates a modification example of the circuit configuration of the measurement electrode module of FIG. 2.

FIG. 15A illustrates a modification example of the circuit configurations of the reference electrode module of FIG. 3A and the measurement electrode module of FIG. 2.

FIG. 15B illustrates a modification example of the circuit configurations of the reference electrode module of FIG. 3B and the measurement electrode module of FIG. 2.

FIG. 16 illustrates a modification example of the circuit configuration of the measurement electrode module of FIG. 2.

FIG. 17 illustrates a modification example of the schematic configuration of the biological information measurement apparatus of FIG. 1.

FIG. 18 illustrates a modification example of the circuit configuration of the measurement electrode module of FIG. 2.

FIG. 19 illustrates a modification example of the circuit configuration of the measurement electrode module of FIG. 10.

FIG. 20 illustrates a modification example of the circuit configuration of the measurement electrode module of FIG. 11.

FIG. 21 illustrates a modification example of the circuit configuration of the measurement electrode module of FIG. 13.

FIG. 22 illustrates a modification example of the circuit configuration of the measurement electrode module of FIG. 14.

FIG. 23 illustrates a modification example of the circuit configuration of the measurement electrode module.

FIG. 24 illustrates a modification example of the number of electrodes inside the measurement electrode module and the reference electrode module.

### MODES FOR CARRYING OUT THE INVENTION

Hereinafter, description is given in detail, with reference to the drawings, of embodiments for carrying out the present disclosure. It is to be noted that the description is given in the following order.

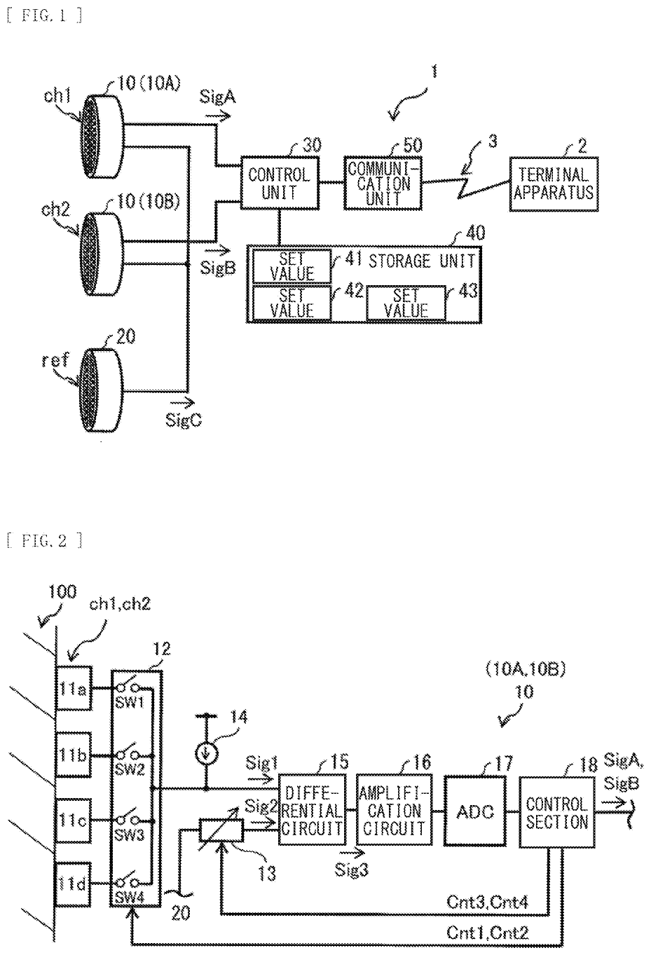

1. Embodiment (Biological Information Measurement Apparatus) . . . FIGS. 1 to 9
An example of performing impedance matching using DC current

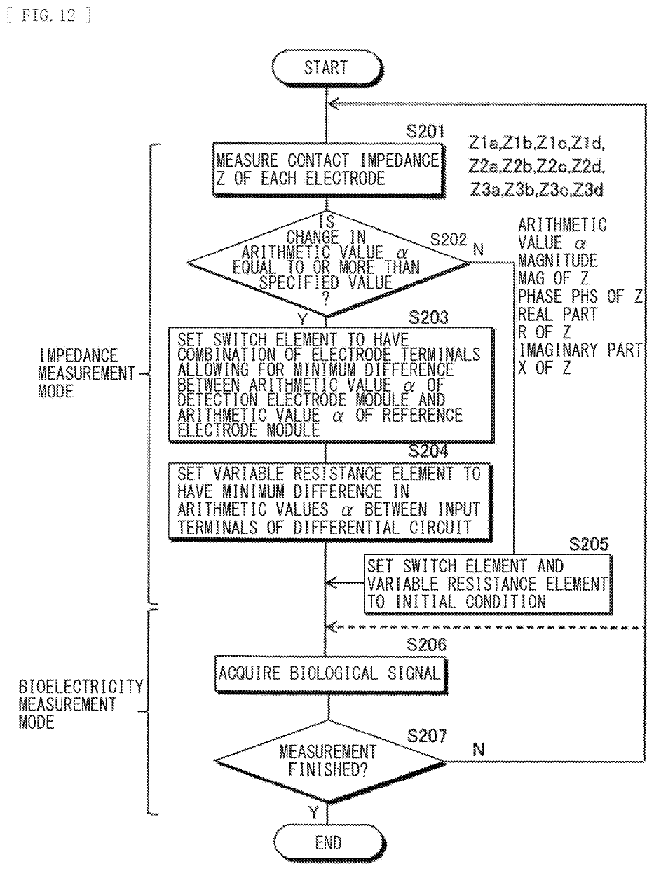

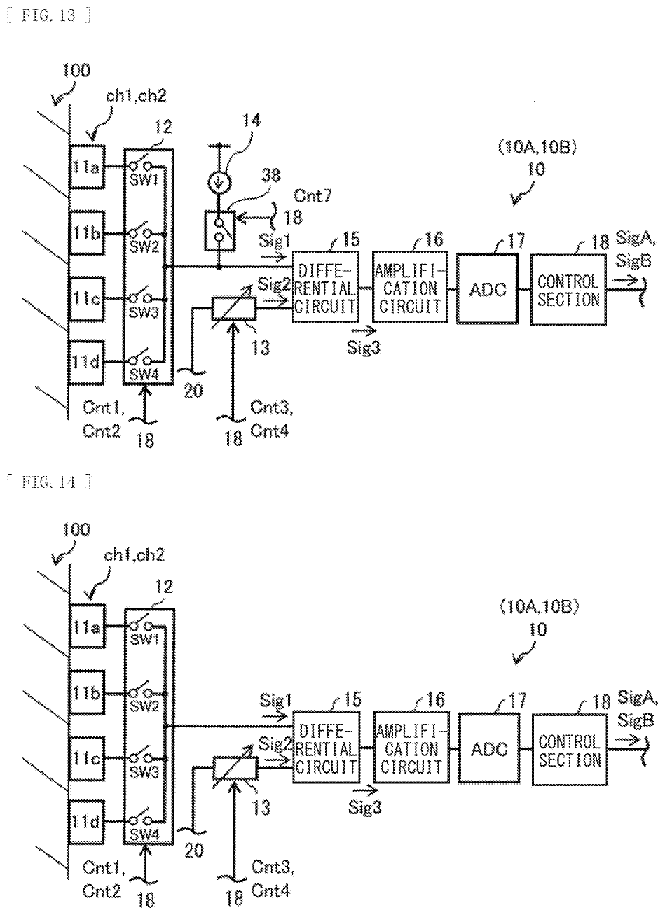

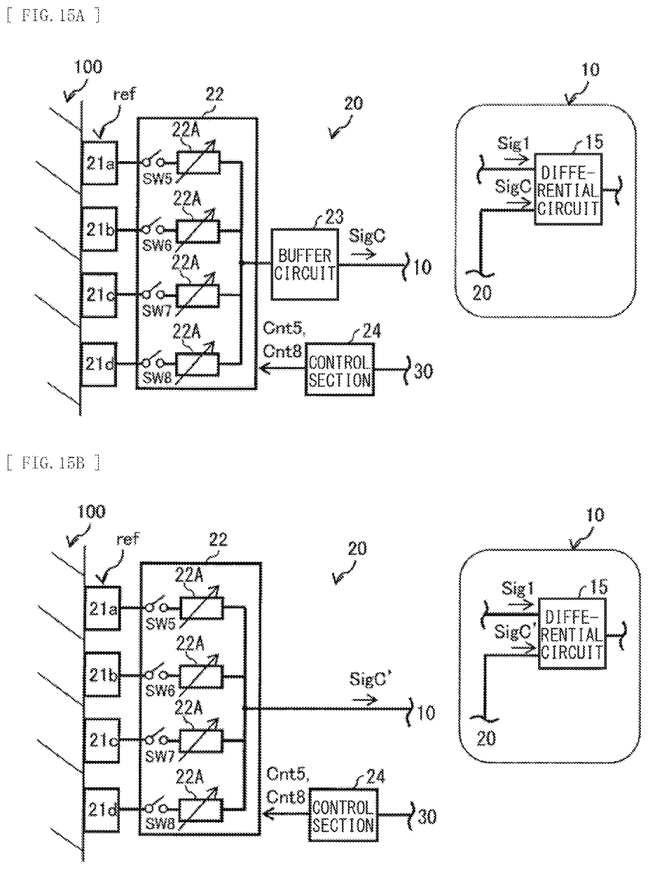

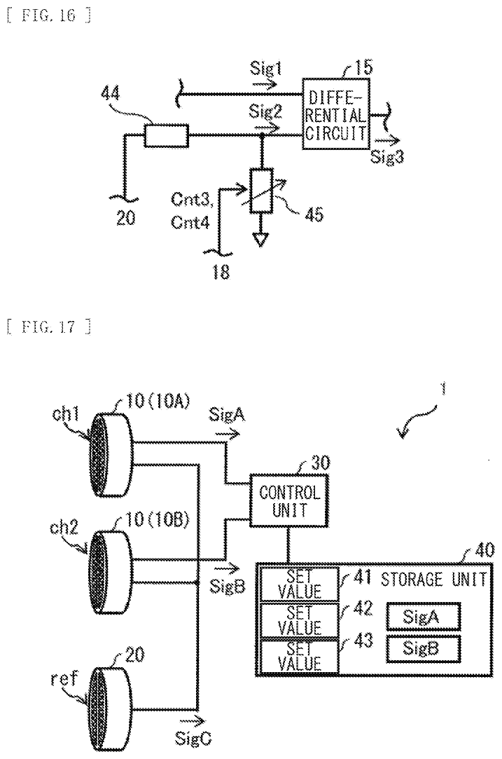

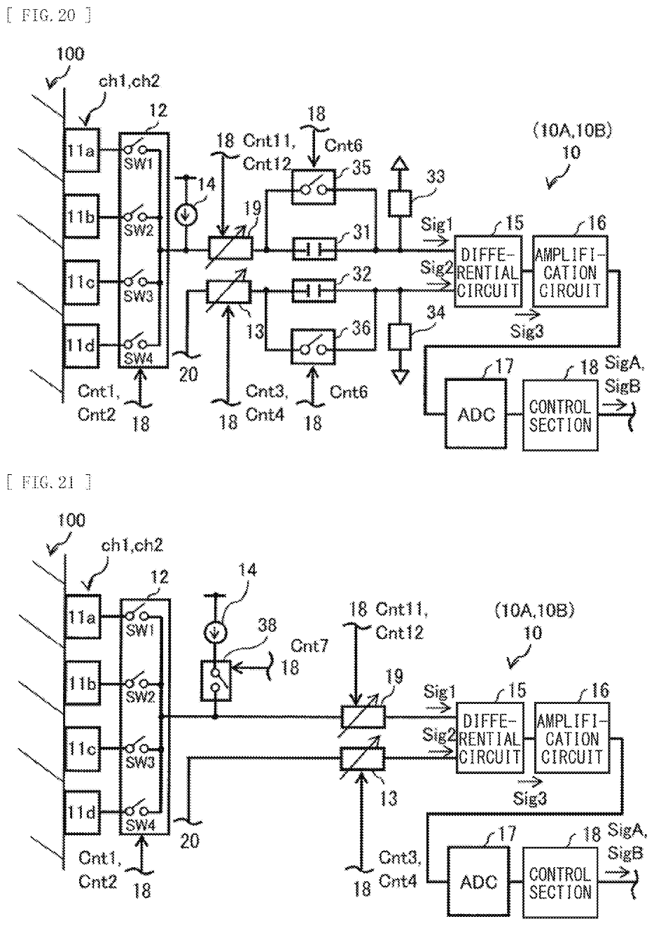

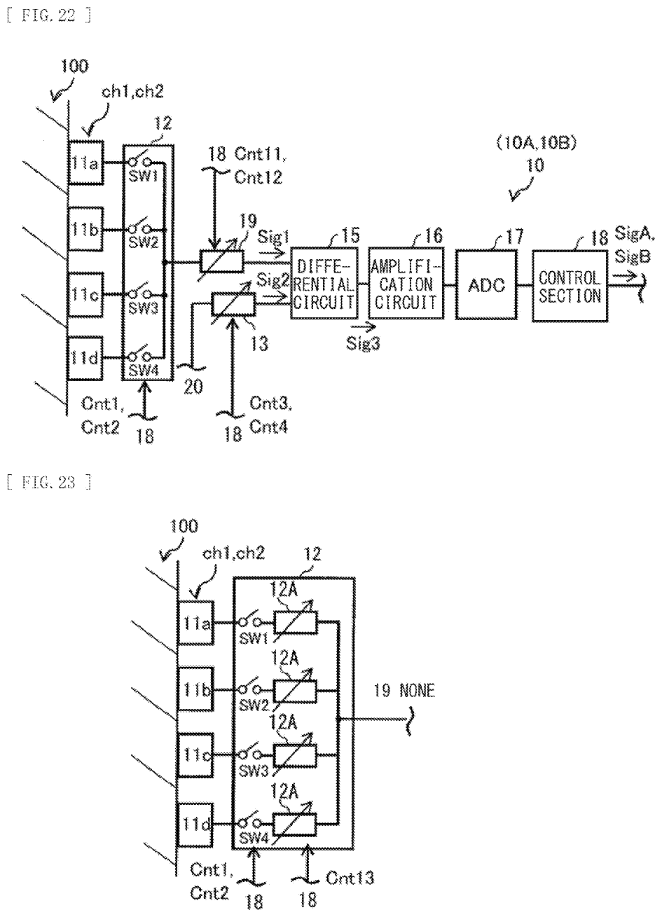

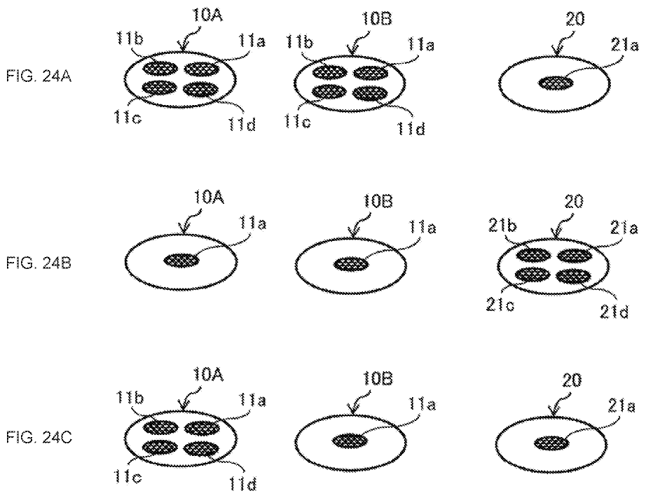

2. Modification Examples (Biological Information Measurement Apparatus)
Modification Example A: An example of using AC coupling circuit . . . FIG. 10
Modification Example B: An example of using AC measurement and DC measurement selectively . . . FIGS. 11 and 12
Modification Example C: An example of turning ON and OFF coupling of a current source . . . FIG. 13
Modification Example D: An example of omitting a current source . . . FIG. 14
Modification Example E: An example of providing a variable resistance element inside a reference electrode module . . . FIG. 15
Modification Example F: An example of voltage dividing of a reference signal between a variable resistance element and a resistance element . . . FIG. 16
Modification Example G: An example of storing a biological signal in a storage unit . . . FIG.
Modification Example H: An example of providing a variable resistance element at both input ends of a differential circuit . . . FIGS. 18 to 22
Modification Example I: An example of providing a variable resistance element inside a measurement electrode module . . . FIG. 23
Modification Example J: A variation of the number of measurement electrodes inside a measurement electrode module and the number of reference electrodes inside a reference electrode module . . . FIG. 24

### 1. EMBODIMENT

### Configuration

Description is given of a biological information measurement apparatus 1 according to an embodiment of the present disclosure. FIG. 1 illustrates an example of a schematic configuration of the biological information measurement apparatus 1. The biological information measurement apparatus 1 is an apparatus that detects biological information of a biological body 100. Examples of the biological information include a brain wave, an electrocardiogram, an electro-oculogram, and the like. The biological body 100 is typically a human, but may also be an animal. The biological information measurement apparatus 1 is, for example, a wearable apparatus such as a head-mounted display.

The biological information measurement apparatus 1 is coupled to a network 3. The network 3 is, for example, a communication line such as LAN or WAN. A terminal apparatus 2 is coupled to the network 3. The biological information measurement apparatus 1 is configured to be able to communicate with the terminal apparatus 2 via the network 3. The terminal apparatus 2 is, for example, a mobile terminal, and is configured to be able to communicate with the biological information measurement apparatus 1 via the network 3.

The terminal apparatus 2 includes an input unit, a control unit, a display unit, and a communication unit. The input unit accepts input information from a user. The control unit transmits the input information inputted to the input unit to the biological information measurement apparatus 1 via the communication unit. The communication unit receives image data from the biological information measurement apparatus 1 via the network 3. The control unit generates an image signal on the basis of the image data received by the communication unit, and outputs the generated image data to the display unit. The display unit displays the image data on the basis of the image signal inputted from the control unit.

The biological information measurement apparatus 1 includes, for example, two measurement electrode modules 10 (10A and 10B), a reference electrode module 20, a control unit 30, a storage unit 40, and a communication unit 50. The number of the measurement electrode modules 10 provided in the biological information measurement apparatus 1 is not limited to two, and may be one or three or more. Hereinafter, description is given on the assumption that the number of the measurement electrode modules 10 provided in the biological information measurement apparatus 1 is two.

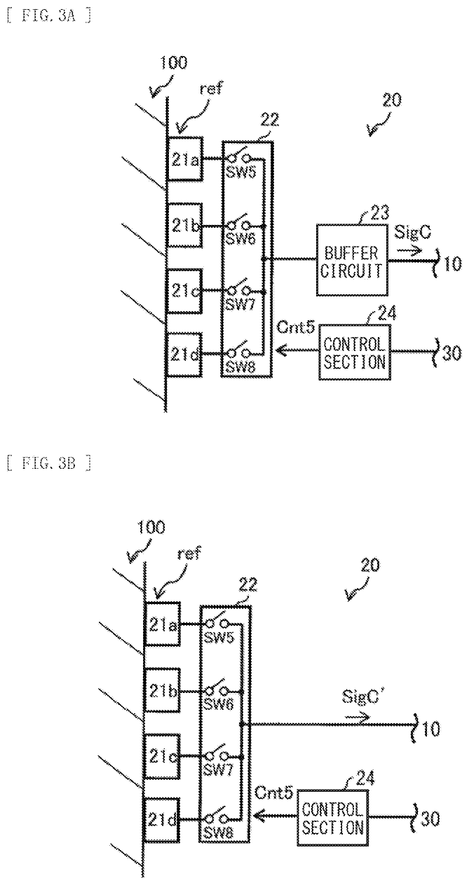

FIG. 2 illustrates a circuit configuration example of each of the measurement electrode modules 10 (10A and 10B). The measurement electrode module 10A includes a plurality of (e.g., four) measurement electrodes 11 (11a, 11b, 11c, and 11d) as a measurement channel ch1 to be in contact with the biological body 100. The measurement electrode module 10B includes a plurality of (e.g., four) measurement electrodes 11 (11a, 11b, 11c, and 11d) as a measurement channel ch2 to be in contact with the biological body 100. The measurement electrodes 11 (11a, 11b, 11c, and 11d) are each a dry electrode to be in contact with the skin of the biological body 100 in a dry environment. The number of the measurement electrodes 11 provided in each of the measurement electrode modules 10 (10A and 10B) is not limited to four, and may be one, two, three, or five or more. Hereinafter, description is given on the assumption that the number of the measurement electrodes 11 provided in each of the measurement electrode modules 10 (10A and 10B) is four.

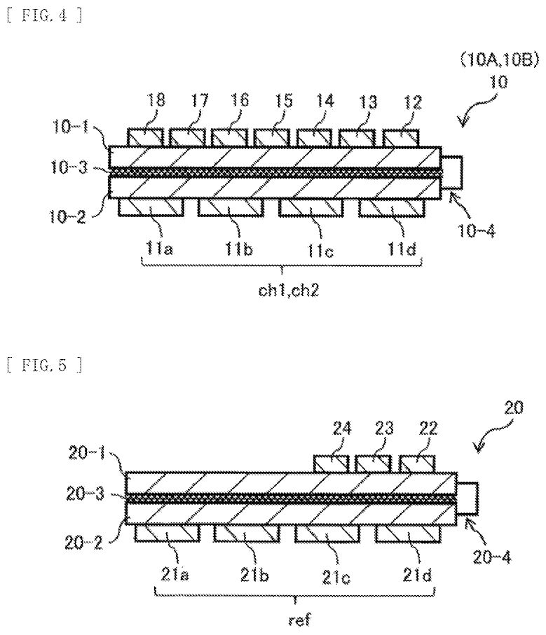

FIG. 3A illustrates an example of a circuit configuration of the reference electrode module 20. The reference electrode module 20 includes a plurality of (e.g., four) reference electrodes 21 (21a, 21b, 21c, and 21d) as a reference channel ref to be in contact with the biological body 100. The reference electrodes 21 (21a, 21b, 21c, and 21d) are each a dry electrode to be in contact with the skin of the biological body 100 in a dry environment. The number of the reference electrodes 21 provided in the reference electrode module 20 is not limited to four, and may be one, two, three, or five or more. Hereinafter, description is given on the assumption that the number of the reference electrodes 21 provided in the reference electrode module 20 is four.

The measurement electrode modules 10 (10A and 10B) each further include a switch element 12, a variable resistance element 13, an AC current source 14, a differential circuit 15, an amplification circuit 16, an ADC (Analog-Digital Converter) 17, and a control section 18. Meanwhile, the reference electrode module 20 further includes a switch element 22, a buffer circuit 23, and a control section 24. It is to be noted that, as illustrated in FIG. 3B, the buffer circuit 23 may be omitted, for example. A circuit including the switch elements 12 and 22, the variable resistance element 13, the control section 18, the buffer circuit 23, the control section 24, and the control unit 30 corresponds to a specific example of a “switch mechanism that switches contact impedance between a biological body and each of a measurement channel and a reference channel” of the present disclosure.

In the measurement electrode module 10A, the switch element 12 selects at least one of the plurality of measurement electrodes 11 (11a, 11b, 11c, and 11d) provided as the measurement channel ch1, on the basis of a control signal Cnt1 from the control section 18. In the measurement electrode module 10A, the switch element 12 is used for adjustment of contact impedance between the biological body 100 and each of the measurement channel ch1 and the reference channel ref. In the measurement electrode module 10B, the switch element 12 selects at least one of the plurality of measurement electrodes 11 (11a, 11b, 11c, and 11d) provided as the measurement channel ch2, on the basis of a control signal Cnt2 from the control section 18. In the measurement electrode module 10B, the switch element 12 is used for adjustment of contact impedance between the biological body 100 and each of the measurement channel ch2 and the reference channel ref.

The switch element 12 includes a plurality of (e.g., four) switches (e.g., switches SW1, SW2, SW3, and SW4) coupled in series one by one for the respective measurement electrodes 11. Hereinafter, description is given on the assumption that the number of the switches provided in the switch element 12 is four. Turning the switches SW1, SW2, SW3, and SW4 ON and OFF is performed on the basis of the control signals Cnt1 and Cnt2 from the control section 18.

In the reference electrode module 20, the switch element 22 selects at least one of the plurality of reference electrodes 21 (21a, 21b, 21c, and 21d) provided as the reference channel ref, on the basis of a control signal Cnt5 from the control section 24. The switch element 22 includes a plurality of (e.g., four) switches (e.g., switches SW5, SW6, SW7, and SW8) coupled in series one by one for the respective reference electrodes 21. Hereinafter, description is given on the assumption that the number of switches provided in the switch element 22 is four. Turning the switches SW5, SW6, SW7, and SW8 ON and OFF is performed on the basis of the control signal Cnt5 from the control section 24.

The buffer circuit 23 is configured by a voltage follower, for example, and performs impedance conversion. An output end of the buffer circuit 23 is electrically coupled to an input end of the differential circuit 15 of each of the measurement electrode modules 10. This suppresses variation in a voltage value of a signal (a reference signal SigC) after the impedance conversion by the buffer circuit 23 depending on the number of the differential circuits 15 coupled to the output end of the buffer circuit 23. In an impedance measurement mode, the control section 24 controls the switch element 22 on the basis of a control signal from the control unit 30 to thereby switch contact impedance between the reference channel ref and the biological body 100. In the impedance measurement mode, the control section 24 further controls the variable resistance element 13 on the basis of a control signal from the control unit 30 to thereby adjust an impedance difference between input terminals of the differential circuit 15.

In the measurement electrode module 10A, the variable resistance element 13 is provided between the plurality of reference electrodes 21 and the differential circuit 15. Specifically, the variable resistance element 13 is inserted in series in a wiring line between the output end of the buffer circuit 23 and the input end (a second input end) of the differential circuit 15. The variable resistance element 13 is used for adjustment of the impedance difference between the input terminals of the differential circuit 15. In the measurement electrode module 10A, a resistance value of the variable resistance element 13 is set on the basis of a control signal Cnt3 from the control section 18. In the measurement electrode module 10A, a resistance value of the variable resistance element 13 is set on the basis of a control signal Cnt4 from the control section 18.

The AC current source 14 is coupled to a wiring line between an output end of the switch element 12 and the input end (a first input end) of the differential circuit 15. The AC current source 14 supplies an AC current to the measurement channels ch1 and ch2. The AC current source 14 is used for measurement of contact impedance between the biological body 100 and each of the measurement channels ch1 and ch2 and the reference channel ref.

In the measurement electrode module 10A, the differential circuit 15 generates a biological signal Sig3 corresponding to a difference between a measurement signal Sig1 obtained from the measurement channel ch1 and a reference signal Sig2 obtained from the reference channel ref. In addition, in the measurement electrode module 10B, the differential circuit 15 generates the biological signal Sig3 corresponding to a difference between the measurement signal Sig1 obtained from the measurement channel ch2 and the reference signal Sig2 obtained from the reference channel ref. In the differential circuit 15, the two input ends are coupled to the output end of the switch element 12 and the variable resistance element 13. The use of the reference signal Sig2 allows the differential circuit 15 to remove a common mode noise (alternate noise) included in the measurement signal Sig1.

The amplification circuit 16 amplifies the biological signal Sig3 inputted from the differential circuit 15. An ADC 17 converts the biological signal Sig3 inputted from the amplification circuit 16 from an analog signal to a digital signal, and outputs the digital biological signal Sig3 to the control section 18.

In the measurement electrode module 10A, the control section 18 performs predetermined processing on the biological signal Sig3, and outputs a biological signal SigA obtained thereby to the control unit 30. In the measurement electrode module 10B, the control section 18 performs predetermined processing on the biological signal Sig3, and outputs a biological signal SigB obtained thereby to the control unit 30.

In the measurement electrode module 10A, the control section 18 controls the switch element 12 on the basis of the control signal from the control unit 30 in the impedance measurement mode to thereby switch contact impedance between the measurement channel ch1 and the biological body 100. In the measurement electrode module 10B, the control section 18 controls the switch element 12 on the basis of the control signal from the control unit 30 in the impedance measurement mode to thereby switch contact impedance between the measurement channel ch2 and the biological body 100. In the measurement electrode modules 10A and 10B, the control section 18 further controls the variable resistance element 13 on the basis of the control signal from the control unit 30 in the impedance measurement mode to thereby switch the impedance difference between the input terminals of the differential circuit 15.

In the measurement electrode module 10A, the control section 18 controls the switch element 12 on the basis of a set value 41 read from the storage unit 40 in a bioelectricity measurement mode to thereby set the contact impedance between the measurement channel ch1 and the biological body 100 to a predetermined value. In the measurement electrode module 10B, the control section 18 controls the switch element 12 on the basis of a set value 42 read from the storage unit 40 in the bioelectricity measurement mode to thereby set the contact impedance between the measurement channel ch2 and the biological body 100 to a predetermined value. In the measurement electrode modules 10A and 10B, the control section 18 further controls the variable resistance element 13 on the basis of a set value 43 read from the storage unit 40 in the bioelectricity measurement mode to thereby set the impedance difference between the input terminals of the differential circuit 15 to a predetermined value.

The control unit 30 generates predetermined image data on the basis of the biological signals SigA and SigB obtained by the measurement electrode modules 10A and 10B. The communication unit 50 transmits the image data generated by the control unit 30 to the terminal apparatus 2 via the network 3. The storage unit 40 stores, for example, the set value 41 (a first set value) of the switch element 12 of each of the measurement electrode modules 10A and 10B, the set value 42 (first set value) of the switch element 22 of the reference electrode module 20, and the set value 43 (a second set value) of the variable resistance element 13 of each of the measurement electrode modules 10A and 10B. The control unit 30 further outputs a control signal to the control section 18 of each of the measurement electrode modules 10A and 10B and the control section 24 of the reference electrode module 20 to thereby control the switch element 22 and the variable resistance element 13 of each of the measurement electrode modules 10A and 10B and the switch element 22 of the reference electrode module 20.

FIG. 4 illustrates a cross-sectional configuration example of the measurement electrode modules 10 (10A and 10B). The measurement electrode modules 10 (10A and 10B) each include, on a wiring substrate 10-1, for example, the switch element 12, the variable resistance element 13, a DC current source 14, the differential circuit 15, the amplification circuit 16, the ADC 17, and the control section 18. The measurement electrode modules 10 (10A and 10B) each further include, on a wiring substrate 10-2, for example, the plurality of measurement electrodes 11 (11a, 11b, 11c, and 11d). The wiring substrate 10-2 is attached to side of a back surface of the wiring substrate 10-1, with a back surface of the wiring substrate 10-2 being opposed thereto. The measurement electrode modules 10 (10A and 10B) may each include, between the wiring substrate 10-1 and the wiring substrate 10-2, for example, a shield layer 10-3 that shields an electric field. The shield layer 10-3 is configured by, for example, a metal thin film. The measurement electrode modules 10 (10A and 10B) each include, for example, a coupling wiring line 10-4 that electrically couples the plurality of measurement electrodes 11 (11a, 11b, 11c, and 11d) on the wiring substrate 10-1 and the switch element 12 on the wiring substrate 10-2 to each other. The coupling wiring line 10-4 may be provided around the shield layer 10-3 or may be provided to penetrate an opening provided in the shield layer 10-3.

FIG. 5 illustrates a cross-sectional configuration example of the reference electrode module 20. The reference electrode module 20 includes, on a wiring substrate 20-1, for example, the switch element 22, the buffer circuit 23, and the control section 24. It is to be noted that the buffer circuit 23 may be omitted. The reference electrode module 20 further includes, on a wiring substrate 20-2, for example, the plurality of reference electrodes 21 (21a, 21b, 21c, and 21d). The wiring substrate 20-2 is attached to side of a back surface of the wiring substrate 20-1, with a back surface of the wiring substrate 20-2 being opposed thereto. The reference electrode module 20 may include, between the wiring substrate 20-1 and the wiring substrate 20-2, for example, a shield layer 20-3 that shields an electric field. The shield layer 20-3 is configured by, for example, a metal thin film. The reference electrode module 20 includes, for example, a coupling wiring line 20-4 that electrically couples the plurality of reference electrodes 21 (21a, 21b, 21c, and 21d) on the wiring substrate 20-1 and the switch element 22 on the wiring substrate 20-2 to each other. The coupling wiring line 20-4 may be provided around the shield layer 20-3 or may be provided to penetrate an opening provided in the shield layer 20-3.

FIG. 6 illustrates a perspective configuration example of the measurement electrode module 10 and the reference electrode module 20. The measurement electrode module 10 and the reference electrode module 20 each have a disk shape. The measurement electrode module 10 includes the plurality of measurement electrodes 11 (11a, 11b, 11c, and 11d) on one surface of the disk (e.g., a front surface of the wiring substrate 10-1). The reference electrode module 20 includes the plurality of reference electrodes 21 (21a, 21b, 21c, and 21d) on one surface of the disk (e.g., a front surface of the wiring substrate 20-2).

The measurement electrode 11 and the reference electrode 21 each have a configuration in which a front surface of copper is plated with silver, for example. In the measurement electrode 11 and the reference electrode 21, the silver plating of the front surface may be chlorinated with a solution, or the like containing sodium chloride. The substrate used for the wiring substrates 10-1,10-2, 20-1, and 20-2 is configured by, for example, a thermoplastic resin such as PVC (polyvinyl chloride), PP (polypropylene), PE (polyethylene), PU (polyurethane), POM (polyacetal), PA (polyamide), PC (polycarbonate), and a copolymer thereof.

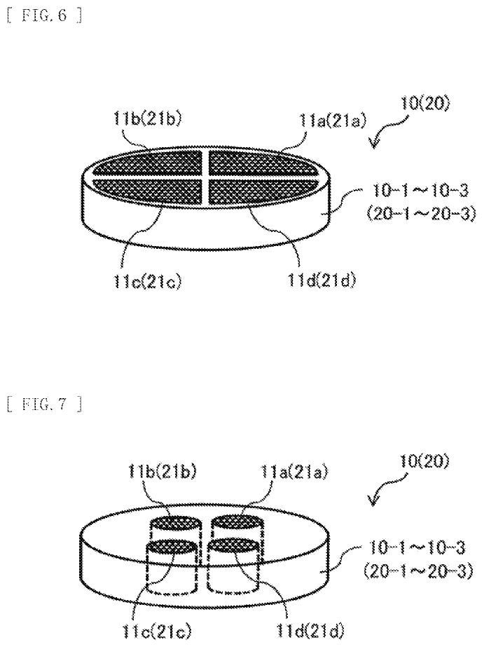

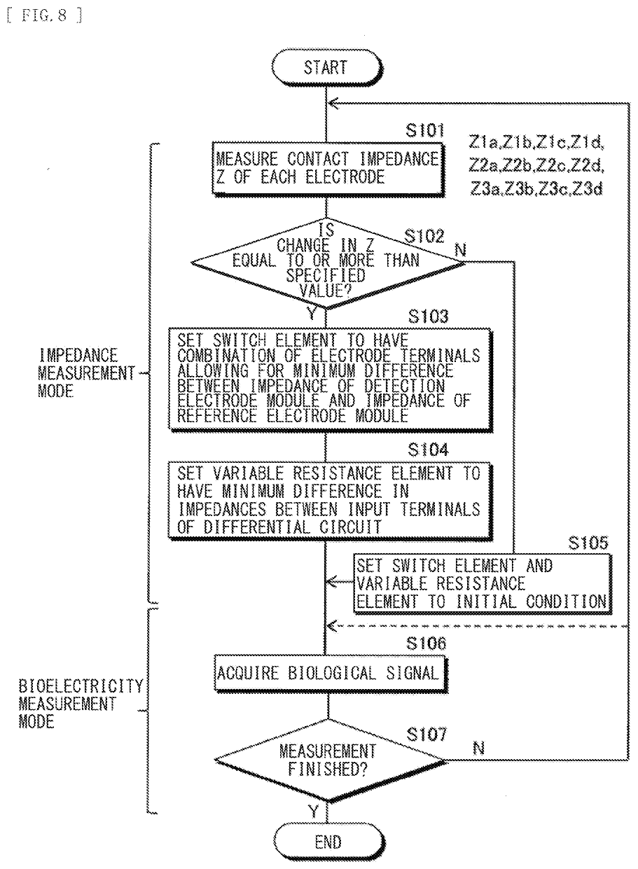

It is to be noted that, as illustrated in FIG. 7, the substrate used for the wiring substrates 10-1, 10-2, 20-1, and 20-2 may be formed by injection molding of an elastomer resin. In this case, the substrate used for the wiring substrates 10-1, 10-2, 20-1, and 20-2 is configured by, for example, a thermosetting elastomer resin such as a silicone resin or a polyurethane resin. At this time, the measurement electrode 11 and the reference electrode 21 may be formed, for example, by molding of a product obtained by kneading electrically-conductive particles such as carbon black into an elastomer resin. The elastomer resin used for the measurement electrode 11 and the reference electrode 21 is preferably an elastomer resin having the same skeleton as that of the elastomer resin used for the wiring substrates 10-1, 10-2, 20-1, and 20-2. As the electrically-conductive particles to be kneaded into the elastomer resin, it is possible to use, in addition to the carbon black, graphite-based particles such as Ketjen black, nanocarbon particles such as a fullerene-carbon nanotube, carbon-based material particles such as graphene particles, particles such as gold, silver and copper, and a nano wire. As the electrically-conductive particles to be kneaded into the elastomer resin, it is preferable to use a material that is able to reduce the contact impedance with the biological body 100. Examples of such a material include a metal compound such as AgCl and Cus, a metal oxide such as PdO₂ and ITO, and electrically-conductive polymer particles and fibers such as PEDOT-PSS, PEDOT-TsO, or polyaniline. As the electrically-conductive particles to be kneaded into the elastomer resin, it is also possible to use a mixture of a plurality of materials from among the above-mentioned materials.

Next, description is given of a measurement procedure in the biological information measurement apparatus 1. FIG. 8 illustrates an example of procedures of impedance matching and biological signal acquisition in the biological information measurement apparatus 1.

### Impedance Measurement Mode

First, the control unit 30 sets the mode to the impedance measurement mode, and starts measuring contact impedance Z of each electrode (step S101). The control unit 30 first instructs the control section 18 of the measurement electrode module 10A and the control section 24 of the reference electrode module 20 to sequentially switch the switch elements 12. On the basis of the instruction from the control unit 30, the control section 18 of the measurement electrode module 10A outputs the control signal Cnt1 to the switch element 12 to thereby control ON and OFF of the plurality of switches SW1, SW2, SW3, and SE4. On the basis of the control signal Cnt1 from the control section 18, the switch element 12 sequentially executes all combinations of ON and OFF of all the switches SW1, SW2, SW3, and SE4 included in the switch element 12. On the basis of the instruction from the control unit 30, the control section 24 of the reference electrode module 20 outputs the control signal Cnt5 to the switch element 22 to thereby control ON and OFF of the plurality of switches SW5, SW6, SW7, and SE8. On the basis of the control signal Cnt5 from the control section 24, the switch element 22 sequentially executes all combinations of ON and OFF of all the switches SW5, SW6, SW7, and SE8 included in the switch element 22, each time switching of the switches inside the switch element 12 is performed.

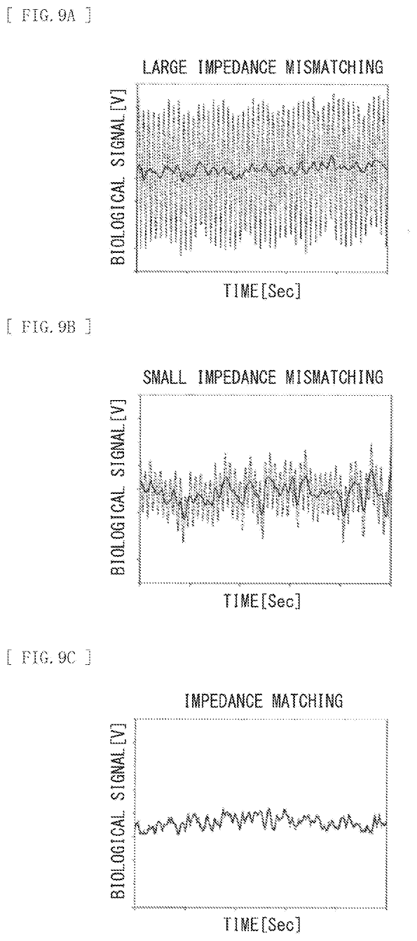

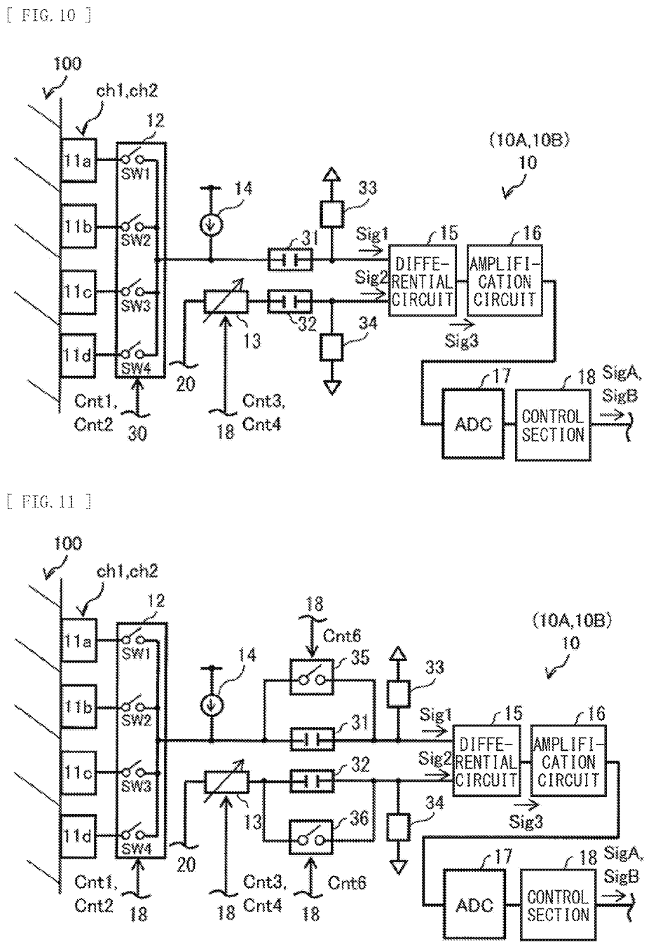

Each time the switching of the switch elements 22 is performed, the differential circuit 15 of the measurement electrode module 10A generates the biological signal Sig3, which is a difference between the measurement signal Sig1 and the reference signal Sig2, and outputs the biological signal Sig3 to the amplification circuit 16. The amplification circuit 16 amplifies the inputted biological signal Sig3, and outputs the amplified biological signal Sig3 to the ADC 17. The ADC 17 converts the analog biological signal Sig3 into a digital biological signal Sig3, and outputs the digital biological signal Sig3 to the control section 18. The control section 18 performs predetermined processing on the biological signal Sig3, and outputs the biological signal SigA obtained thereby to the control unit 30. The control unit 30 generates predetermined image data on the basis of the biological signal SigA. The communication unit 50 transmits the image data generated by the control unit 30 to the terminal apparatus 2 via the network 3. The terminal apparatus 2 displays, on a display unit, the image data received from the biological information measurement apparatus 1. At this time, the display unit displays, for example, a signal waveform including the biological signal as illustrated in FIGS. 9A and 9B.

Next, on the basis of each biological signal Sig3, the control unit 30 calculates contact impedances Z (Z1a, Z1b, Z1c, and Z1d) between the biological body 100 and each of the measurement electrodes 11 (11a, 11b, 11c, and 11d) of the measurement electrode module 10A, and contact impedances Z (Z3a, Z3b, Z3c, and Z3d) between the biological body 100 and each of the reference electrodes 21 (21a, 21b, 21c, and 21d) of the reference electrode module 20. Subsequently, the control unit 30 determines whether or not a change in the calculated plurality of contact impedances Z is equal to or more than a specified value (step S102). As a result, in a case where the change in the calculated plurality of contact impedances Z is equal to or more than the specified value, the control unit 30 derives set values of the switch elements 12 and 22 corresponding to a combination of electrodes having the minimum difference between the plurality of contact impedances Z (Z1a, Z1b, Z1c, and Z1d) of the measurement electrode module 10A and the plurality of contact impedances Z (Z3a, Z3b, Z3c, and Z3d) of the reference electrode module 20 (step S103). The control unit 30 transmits, for example, the derived set value and the signal waveform corresponding to the set value to the terminal apparatus 2 via the network 3. The terminal apparatus 2 displays, on the display unit, the set value and the signal waveform received from the biological information measurement apparatus 1.

Suppose, at this time, that the signal waveform presented newly by the control unit 30 is, for example, a signal waveform as illustrated in FIG. 9C. In a case where the common mode noise included in the biological signal SigA is small enough to be almost invisible in this manner, it is presumed that the impedance difference between the input terminals of the differential circuit 15 is very small. However, suppose that the image data presented newly by the control unit 30 is, for example, a signal waveform as illustrated in FIG. 9B. In a case where the common mode noise included in the biological signal SigA is large enough to be sufficiently visible in this manner, it is presumed that the impedance difference between the input terminals of the differential circuit 15 is not sufficiently small. In any case, when the common mode noise included in the biological signal SigA inside the image data presented newly by the control unit 30 is smaller than the common mode noise included in the biological signal SigA inside another image data, the set values 41 and 42 derived by the control unit 30 are presumed to be values that are able to effectively reduce the common mode noise included in the biological signal SigA even in a case where the contact status of the electrode is changed due to body movement or insufficient attachment. Therefore, the user operates the input unit of the terminal apparatus 2 to thereby choose to set the set values of the switch elements 12 and 22 to set values corresponding to the image data presented newly by the control unit 30. It is to be noted that, in a case where the common mode noise included in the biological signal SigA inside the signal waveform presented newly by the control unit 30 is larger than the common mode noise included in the biological signal SigA inside the other image data, the user operates the input unit of the terminal apparatus 2 to thereby choose to set the set values of the switch elements 12 and 22 to set values corresponding to image data with the smallest common mode noise included in the biological signal SigA.

The terminal apparatus 2 transmits the set values inputted via the input unit to the biological information measurement apparatus 1 via the communication unit and the network 3. The biological information measurement apparatus 1 (control unit 30) stores, in the storage unit 40, the set values inputted from the terminal apparatus 2, as the set values 41 and 42 of the switch elements 12 and 22. That is, the control unit 30 causes the storage unit 40 to store the set value 41 of the switch element 12 of each of the measurement electrode modules 10A and 10B and the set value 42 of the switch element 22 of the reference electrode module 20, which have been obtained in the impedance measurement mode.

The control unit 30 outputs the set value 41 inputted from the terminal apparatus 2 to the control section 18 of the measurement electrode module 10A. The control unit 30 further outputs the set value 42 inputted from the terminal apparatus 2 to the control section 24 of the reference electrode module 20. The control section 18 of the measurement electrode module 10A outputs the set value 41 inputted from the control unit 30 to the switch element 12, and the control section 24 of the reference electrode module 20 outputs the set value 42 inputted from the control unit 30 to the switch element 22. The switch element 12 sets the switches SW1, SW2, SW3, and SW4 to the set value 41 inputted from the control unit 30 to thereby select at least one of the plurality of measurement electrodes 11 (11a, 11b, 11c, and 11d) provided as the measurement channel ch1. Meanwhile, the switch element 22 sets the switches SW5, SW6, SW7, and SW8 to the set value 42 inputted from the control unit 30 to thereby select at least one of the plurality of reference electrodes 21 (21a, 21b, 21c, and 21d) provided as the reference channel ref.

Next, the control unit 30 controls resistance values of resistors inside the variable resistance element 13 with respect to the control section 18 of the measurement electrode module 10A. On the basis of an instruction from the control unit 30, the control section 18 of the measurement electrode module 10A outputs the control signal Cnt3 to the variable resistance element 13 to thereby control switching of the resistors inside the variable resistance element 13. The variable resistance element 13 sequentially executes all combinations of the resistors inside the variable resistance element 13 on the basis of the control signal Cnt3 from the control section 18.

Each time the switching of the resistors inside the variable resistance element 13 is performed, the differential circuit 15 of the measurement electrode module 10A generates the biological signal Sig3, which is a difference between the measurement signal Sig1 and the reference signal Sig2, and outputs the biological signal Sig3 to the amplification circuit 16. The amplification circuit 16 amplifies the inputted biological signal Sig3, and outputs the amplified biological signal Sig3 to the ADC 17. The ADC 17 converts the analog biological signal Sig3 into the digital biological signal Sig3, and outputs the digital biological signal Sig3 to the control section 18. The control section 18 performs predetermined processing on the biological signal Sig3, and outputs the biological signal SigA obtained thereby to the control unit 30. The control unit 30 generates predetermined image data on the basis of the biological signal SigA. The communication unit 50 transmits the image data generated by the control unit 30 to the terminal apparatus 2 via the network 3. The terminal apparatus 2 displays, on the display unit, the biological signal waveform received from the biological information measurement apparatus 1. At this time, the display unit displays, for example, the signal waveform including the biological signal as illustrated in FIGS. 9B and 9C.

Next, on the basis of each biological signal Sig3, the control unit 30 calculates impedance Za of one input end (first input end) of the differential circuit 15 and impedance Zb of another input end (second input end) of the differential circuit 15. Subsequently, the control unit 30 derives a set value of the variable resistance element 13 corresponding to a combination of the resistors inside the variable resistance element 13 having the minimum difference between the calculated impedances Za and Zb (step S104). The control unit 30 transmits, for example, the derived set value and the image data corresponding to the set value to the terminal apparatus 2 via the network 3. The terminal apparatus 2 displays, on the display unit, the set value and the signal waveform received from the biological information measurement apparatus 1.

At this time, suppose that the image data presented newly by the control unit 30 is, for example, image data as illustrated in FIG. 9C. In a case where the common mode noise included in the biological signal SigA is small enough to be almost invisible in this manner, it is presumed that the impedance difference between the input terminals of the differential circuit 15 is very small. In this case, even when the contact status of the electrode is changed due to body movement or insufficient attachment, the set value derived by the control unit 30 is presumed to be a value that is able to effectively reduce the common mode noise included in the biological signal SigA. Therefore, the user operates the input unit of the terminal apparatus 2 to thereby choose to set the set value of the variable resistance element 13 to a set value corresponding to the image data presented newly by the control unit 30.

The terminal apparatus 2 transmits the set value inputted via the input unit to the biological information measurement apparatus 1 via the communication unit and the network 3. The biological information measurement apparatus 1 (control unit 30) stores, as the set value 43 of the variable resistance element 13, the set value inputted from the terminal apparatus 2. The control unit 30 further outputs the set value 43 inputted from the terminal apparatus 2 to the control section 18 of the measurement electrode module 10A. The control section 18 of the measurement electrode module 10A outputs the set value 43 inputted from the control unit 30 to the variable resistance element 13. The variable resistance element 13 sets the resistor inside the variable resistance element 13 to have the set value 43 inputted from the control unit 30.

It is to be noted that, in a case where the change in the calculated plurality of contact impedances Z is less than the specified value in step S102, the control unit 30 sets the set values of the switch elements 12 and 22 and the variable resistance element 13 to an initial condition (step S105).

### Bioelectricity Measurement Mode

Next, the control unit 30 sets the mode to the bioelectricity measurement mode, and controls the switch elements 12 and 22 and the variable resistance element 13 on the basis of the set values 41, 42, and 43 obtained in the impedance measurement mode. After setting the set values 41, 42, and 43 obtained in the impedance measurement mode for the switch elements 12 and 22 and the variable resistance element 13, the control unit 30 acquires the biological signal SigA from the measurement electrode module 10A, for example, at a predetermined cycle (step S106). That is, when the set values 41, 42, and 43 are set for the switch elements 12 and 22 and the variable resistance element 13 (in the bioelectricity measurement mode), the differential circuit 15 inside the measurement electrode modules 10A generates the biological signal Sig3. On the basis of the biological signal Sig3 thus obtained, the measurement electrode module 10A generates the biological signal SigA, and outputs the generated biological signal SigA to the control unit 30. The control unit 30 generates predetermined image data on the basis of the biological signal SigA obtained by the measurement electrode modules 10A and 10B. The communication unit 50 transmits the image data generated by the control unit 30 to the terminal apparatus 2 via the network 3. The terminal apparatus 2 displays, on the display unit, the image data inputted from the biological information measurement apparatus 1. In this manner, the biological signal obtained in the bioelectricity measurement mode is displayed on the display unit of the terminal apparatus 2.

The control unit 30 finishes the measurement in a case where an instruction to finish the measurement is inputted from the terminal apparatus 2, and continues to acquire the biological signal SigA or repeats the procedure from step S01 in a case where the instruction to finish the measurement is not inputted from the terminal apparatus 2 (step S107).

It is to be noted that procedures of impedance matching and biological signal acquisition using the measurement electrode module 10B are similar to the above-described procedures of the impedance matching and the biological signal acquisition using the measurement electrode module 10A. In this manner, the impedance matching and the biological signal acquisition in the biological information measurement apparatus 1 are performed.

### Effects

Next, description is given of effects of the biological information measurement apparatus 1.

Noises occurring as a result of an external electromagnetic wave being coupled to and mixed into a human body or a wiring line have become a major issue in an apparatus that measures a minute electric potential difference such as a brain wave. Among such alternate noises, a noise as a result of electrostatic induction with the human body is usually reduced by a differential circuit. However, when a difference occurs in contact impedances between the biological body and two electrodes coupled to the differential circuit, the alternate noise remains without being able to be removed by the differential circuit. A magnitude of the alternate noise is known to be proportional to the difference in the contact impedances.

In order to reduce such a problem, electroencephalographs for research and medical applications undergo a measure of increasing input impedance of a differential amplifier. In a measurement environment assumed by the electroencephalograph, such an electrode as to reduce contact impedance with a biological body is utilized with the use of a measuring gel, a physiological saline solution, or the like, which is generally called a wet electrode; the magnitude of the contact impedance is about several kΩ, and measurement is performed under such a circumstance where such an artifact as to largely change the contact impedance does not occur. In such an environment, the alternate noise has not been a major issue.

However, in a consumer application, it is difficult to use the wet electrode from the viewpoint of a taint on a user with a gel, a physiological saline solution, etc., occurrence of a chronological change in the gel, the physiological saline solution, etc., troublesomeness in using the gel, the physiological saline, or the like. Therefore, in the consumer application, it is considered necessary to use a dry-type electrode called a dry electrode. Although the dry electrode is able to be easily attached, the contact impedance is as large as 10 kΩ to 1 MΩ, and variation among measurement sites (electrodes) is also large. In addition, an assumed usage condition is a daily life, and thus the contact impedance between the electrode and biological body largely is changed dynamically due to an influence of body movement. As described above, in such a circumstance, the removal of the alternate noise by the differential circuit becomes insufficient, thus significantly deteriorating the quality of measurement, which has been an issue.

In addition, in a case where the alternate noise is included in a biological signal, it is necessary to secure a large dynamic range in a differential circuit, an amplification circuit, and an ADC, as compared with a case where no alternate noise is included. When the dynamic range is insufficient, saturation occurs in the differential circuit, the amplification circuit, and the ADC, thus making it hardly possible to obtain an accurate biological signal during the saturation. Therefore, significant deterioration in the quality of measurement has been an issue.

Meanwhile, in the present embodiment, the differential circuit 15 is provided that generates the biological signal Sig3 corresponding to the difference between the measurement signal Sig1 and the reference signal Sig2, and a switch mechanism (a circuit including the switch elements 12 and 22, the variable resistance element 13, the control section 18, the buffer circuit 23, the control section 24, and the control unit 30) is further provided that switches the contact impedance between the biological body 100 and each of the measurement channels ch1 and ch2 and the reference channel ref. This enables adjustment of the contact impedance depending on the contact status of each of the measurement channels ch1 and ch2 and the reference channel ref. As a result, it is possible to effectively reduce the alternate noise included in the biological signal Sig3 even under a circumstance where the contact status of each of the measurement channels ch1 and ch2 and the reference channel ref may be changed.

In addition, in the present embodiment, the measurement channels ch1 and ch2 each include the plurality of measurement electrodes 11, and the reference channel ref includes the plurality of reference electrodes 21. Further, the switch element 12 that selects at least one of the plurality of measurement electrodes 11 and the switch element 22 that selects at least one of the plurality of reference electrodes 21 are provided; controlling the switch elements 12 and 22 allows for switching of the contact impedance. This enables adjustment of the contact impedance depending on the contact status of each of the measurement channels ch1 and ch2 and the reference channel ref. As a result, it is possible to effectively reduce the alternate noise included in the biological signal Sig3 even under a circumstance where the contact status of each of the measurement channels ch1 and ch2 and the reference channel ref may be changed.

In addition, in the present embodiment, the variable resistance element 13 is provided between the plurality of reference electrodes 21 and the differential circuit 15; controlling the variable resistance element 13 allows for switching of the impedance difference between the input terminals of the differential circuit 15. This makes it possible to adjust the impedance difference between the input terminals of the differential circuit 15, depending on the contact status of each of the measurement channels ch1 and ch2 and the reference channel ref. As a result, it is possible to effectively reduce the alternate noise included in the biological signal Sig3 even under a circumstance where the contact status of each of the measurement channels ch1 and ch2 and the reference channel ref may be changed.

In addition, in the present embodiment, control is performed, in the impedance measurement mode, to switch the contact impedance between the biological body 100 and each of the measurement channels ch1 and ch2 and the reference channel ref as well as the impedance difference between the input terminals of the differential circuit 15, thus resulting in acquisition of the set values 41 and 42 of the switch elements 12 and 22 and the set value 43 of the variable resistance element 13. This makes it possible to adjust the impedance difference between the input terminals of the differential circuit 15, depending on the contact status of each of the measurement channels ch1 and ch2 and the reference channel ref. As a result, it is possible to effectively reduce the alternate noise included in the biological signal Sig3 even under a circumstance where the contact status of each of the measurement channels ch1 and ch2 and the reference channel ref may be changed.

In addition, in the present embodiment, control is performed, in the bioelectricity measurement mode, on the switch elements 12 and 22 and the variable resistance element 13 on the basis of the set values 41, 42, and 43 obtained in the impedance measurement mode. This makes it possible to obtain the biological signal Sig3 in which the alternate noise is effectively reduced.

In addition, in the present embodiment, the DC current source 14 is provided that supplies a DC current to the measurement channels ch1 and ch2. This makes it possible to accurately obtain, in the impedance measurement mode, the contact impedance between the biological body 100 and each of the measurement channels ch1 and ch2 and the reference channel ref as well as the impedance difference between the input terminals of the differential circuit 15, as compared with a case where the DC current source 14 is not provided. As a result, it is possible to accurately obtain the set values 41 and 42 of the switch elements 12 and 22 and the set value 43 of the variable resistance element 13. Thus, it is possible to obtain the biological signal Sig3 in which the alternate noise is effectively reduced.

In addition, in the present embodiment, the communication unit 50 is provided that transmits the biological signal Sig3 to the terminal apparatus 2. This makes it possible to reduce a size of the biological information measurement apparatus 1, because there is no need to provide the biological information measurement apparatus 1 with a display unit for confirmation of the biological signal Sig3.

### 2. MODIFICATION EXAMPLES

Next, description is given of modification examples of the biological information measurement apparatus 1 according to the foregoing embodiment.

### Modification Example A

FIG. 10 illustrates a modification example of the circuit configuration of the measurement electrode module 10 provided in the biological information measurement apparatus 1 according to the foregoing embodiment. In the present modification example, the biological information measurement apparatus 1 according to the foregoing embodiment is provided with AC coupling circuits 31 and 32. As bioelectricity measurement, DC measurement using a DC coupling circuit as illustrated in FIG. 2 and AC measurement using an AC coupling circuit as illustrated in FIG. 10 are conceivable. The impedance-switching/adjustment mechanism of the present disclosure is applicable to both the DC measurement system and the AC measurement system; FIG. 10 illustrates a modification example applied to the AC measurement system.

### Modification Example B

FIG. 11 illustrates a modification example of the circuit configuration of the measurement electrode module 10 in the above-described Modification Example A. In the present modification example, the AC coupling circuits 31 and 32 of FIG. 10 are provided with switch elements 35 and 36 in parallel, thus obtaining circuits achieving both of the AC coupling circuit and the DC coupling circuit. The AC measurement and the DC measurement may be selectively used depending on the purpose.

Next, description is given of a measurement procedure in the above-described Modification Examples A and B. FIG. 12 illustrates an example of procedures of impedance matching and biological signal acquisition in the above-described Modification Examples A and B.

### Impedance Measurement Mode

First, the control unit 30 sets the mode to the impedance measurement mode, and starts measuring the contact impedance Z of each electrode (step S201). In a method similar to that of the foregoing embodiment, the control unit 30 calculates the contact impedances Z (Z1a, Z1b, Z1c, and Z1d) between the biological body 100 and each of the measurement electrodes 11 (11a, 11b, 11c, and 11d) of the measurement electrode module 10A, and the contact impedances Z (Z3a, Z3b, Z3c, and Z3d) between the biological body 100 and each of the reference electrodes 21 (21a, 21b, 21c, and 21d) of the reference electrode module 20.

Next, the control unit 30 calculates a predetermined arithmetic value α on the basis of the calculated plurality of contact impedances Z. The predetermined calculated value α is, for example, magnitude MAG of the contact impedance Z, a phase PHS of the contact impedance Z, a real part R of the contact impedance Z, or an imaginary part X of the contact impedance Z.

Subsequently, the control unit 30 determines whether or not a change in the calculated plurality of calculated values α is equal to or more than a specified value (step S202). As a result, in a case where the change in the calculated plurality of calculated values α is equal to or more than the specified value, the control unit 30 derives set values of the switch elements 12 and 22 corresponding to a combination of electrodes having the minimum difference between the plurality of calculated values α of the measurement electrode module 10A and the plurality of calculated values α of the reference electrode module 20 (step S203).

The terminal apparatus 2 transmits a set value selected by a user to the biological information measurement apparatus 1 via the communication unit and the network 3. The biological information measurement apparatus 1 (control unit 30) stores, in the storage unit 40, the set value inputted from the terminal apparatus 2, as the set values 41 and 42 of the switch elements 12 and 22. That is, the control unit 30 causes the storage unit 40 to store the set value 41 of the switch element 12 of each of the measurement electrode modules 10A and 10B, and the set value 42 of the switch element 22 of the reference electrode module 20, which have been obtained in the impedance measurement mode.

The control unit 30 outputs the set value 41 inputted from the terminal apparatus 2 to the control section 18 of the measurement electrode module 10A. The control unit 30 further outputs the set value 42 inputted from the terminal apparatus 2 to the control section 24 of the reference electrode module 20. The control section 18 of the measurement electrode module 10A outputs the set value 41 inputted from the control unit 30 to the switch element 12, and the control section 24 of the reference electrode module 20 outputs the set value 42 inputted from the control unit 30 to the switch element 22. The switch element 12 sets the switches SW1, SW2, SW3, and SW4 to the set value 41 inputted from the control unit 30 to thereby select at least one of the plurality of measurement electrodes 11 (11a, 11b, 11c, and 11d) provided as the measurement channel ch1. Meanwhile, the switch element 22 sets the switches SW5, SW6, SW7, and SW8 to the set value 42 inputted from the control unit 30 to thereby select at least one of the plurality of reference electrodes 21 (21a, 21b, 21c, and 21d) provided as the reference channel ref.

Next, in a method similar to that of the foregoing embodiment, the control unit 30 calculates a calculated value αa of one input end (first input end) of the differential circuit 15, and a calculated value αb of another input end (second input end) of the differential circuit 15. Subsequently, the control unit 30 derives a set value of the variable resistance element 13 corresponding to a combination of resistors inside the variable resistance element 13 having the minimum difference between the calculated values αa and αb that are calculated (step S104).

The terminal apparatus 2 transmits a set value selected by a user to the biological information measurement apparatus 1 via the communication unit and the network 3. The biological information measurement apparatus 1 (control unit 30) stores, as the set value 43 of the variable resistance element 13, the set value inputted from the terminal apparatus 2. The control unit 30 further outputs the set value 43 inputted from the terminal apparatus 2 to the control section 18 of the measurement electrode module 10A. The control section 18 of the measurement electrode module 10A outputs the set value 43 inputted from the control unit 30 to the variable resistance element 13. The variable resistance element 13 sets the resistor inside the variable resistance element 13 to have the set value 43 inputted from the control unit 30.

It is to be noted that, in a case where the change in the calculated plurality of contact impedances Z is less than the specified value in step S102, the control unit 30 sets the set values of the switch elements 12 and 22 and the variable resistance element 13 to an initial condition (step S205).

### Bioelectricity Measurement Mode

Next, the control unit 30 sets the mode to the bioelectricity measurement mode, and controls the switch elements 12 and 22 and the variable resistance element 13 on the basis of the set values 41, 42, and 43 obtained in the impedance measurement mode. After setting the set values 41, 42, and 43 obtained in the impedance measurement mode for the switch elements 12 and 22 and the variable resistance element 13, the control unit 30 acquires the biological signal SigA from the measurement electrode module 10A, for example, at a predetermined cycle (step S106). That is, when the set values 41, 42, and 43 are set for the switch elements 12 and 22 and the variable resistance element 13 (in the bioelectricity measurement mode), the differential circuit 15 inside the measurement electrode modules 10A generates the biological signal Sig3. On the basis of the biological signal Sig3 thus obtained, the measurement electrode module 10A generates the biological signal SigA, and outputs the generated biological signal SigA to the control unit 30. The control unit 30 generates predetermined image data on the basis of the biological signal SigA obtained by the measurement electrode modules 10A and 10B. The communication unit 50 transmits the image data generated by the control unit 30 to the terminal apparatus 2 via the network 3. The terminal apparatus 2 displays, on the display unit, the image data inputted from the biological information measurement apparatus 1. In this manner, the biological signal obtained in the bioelectricity measurement mode is displayed on the display unit of the terminal apparatus 2.

The control unit 30 finishes the measurement in a case where an instruction to finish the measurement is inputted from the terminal apparatus 2, and continues to acquire the biological signal SigA or repeats the procedure from step S01 in a case where the instruction to finish the measurement is not inputted from the terminal apparatus 2 (step S207).

It is to be noted that procedures of impedance matching and biological signal acquisition using the measurement electrode module 10B are similar to the above-described procedures of the impedance matching and the biological signal acquisition using the measurement electrode module 10A. In this manner, the impedance matching and the biological signal acquisition in the biological information measurement apparatus 1 are performed.

From those described above, effects similar to those of the foregoing embodiment are achieved in the above-described Modification Examples A and B even in a case where the arithmetic value α is used.

### Modification Example C

FIG. 13 illustrates a modification example of the circuit configuration of the measurement electrode module 10 in the biological information measurement apparatus 1 according to any of the foregoing embodiment and modification examples thereof. In the present modification example, the biological information measurement apparatus 1 according to any of the foregoing embodiment and modification examples thereof is provided with a switch element 38 between an output end of the AC current source 14 and a wiring line linking the switch element 12 and the one input end (first input end) of the differential circuit 15. The switch element 38 performs connection and disconnection between the AC current source 14 and the switch element 12. In such a case, the control section 18 is able to turn ON the switch element 38 in the impedance measurement mode, and is able to turn ON the switch element 38 in the bioelectricity measurement mode.

This makes it possible to prevent the ADC 17 from being saturated by the AC current, in the bioelectricity measurement mode, because no AC current from the AC current source 14 is inputted to the ADC 17. As a result, it is possible to accurately obtain the set values 41 and 42 of the switch elements 12 and 22 and the set value 43 of the variable resistance element 13, and, in addition, it is possible to obtain the biological signal Sig3 in which the alternate noise is effectively reduced. In addition, it is possible to employ, as the ADC 17, the one with a small bit-depth, thus making it possible to measure bioelectricity with low power consumption.

### Modification Example D

FIG. 14 illustrates a modification example of the circuit configuration of the measurement electrode module 10 in the biological information measurement apparatus 1 according to any of the foregoing embodiment and modification examples thereof. In the present modification example, a current source is omitted in the biological information measurement apparatus 1 according to any of the foregoing embodiment and modification examples thereof. Also in such a case, it is possible to obtain the set values 41 and 42 of the switch elements 12 and 22 and the set value 43 of the variable resistance element 13, and, in addition, it is possible to obtain the biological signal Sig3 in which the alternate noise is effectively reduced.

### Modification Example E

FIG. 15 illustrates a modification example of the circuit configuration of the measurement electrode module 10 and the reference electrode module 20 in the biological information measurement apparatus 1 according to any of the foregoing embodiment and modification examples thereof. In the present modification example, the variable resistance element 13 is omitted in the measurement electrode module 10, and variable resistance elements 22A are provided one by one for the switches SW5, SW6, SW7, and SW8 in the switch element 22 of the reference electrode module 20. In each of the variable resistance elements 22A, ON-OFF control is performed in accordance with a control signal Cnt8 from the control section 24. A method for deriving a set value of each of the variable resistance elements 22A is similar to the method for deriving the set value of the variable resistance element 13 in the foregoing embodiment and modification examples thereof. Thus, also in the present modification example, it is possible to obtain the biological signal Sig3 in which the alternate noise is effectively removed, similarly to the foregoing embodiment and modification examples thereof.

### Modification Example F

FIG. 16 illustrates a modification example of the circuit configuration of the measurement electrode module 10 and the reference electrode module 20 in the biological information measurement apparatus 1 according to any of the foregoing embodiment and modification examples thereof. In the present modification example, a variable resistance element 45 is provided instead of the variable resistance element 13. The variable resistance element 45 is coupled to a wiring line that couples together an output end of the reference electrode module 20 and the input end (second input end) of the differential circuit 15 to allow for branching. In the present modification example, a resistance element 44 is further inserted in series in the wiring line that couples together the output end of the reference electrode module 20 and the input end (second input end) of the differential circuit 15. A voltage to be inputted to the input end (second input end) of the differential circuit 15 is divided by the resistance element 44 and the variable resistance element 45. Also in such a case, it is possible to obtain the biological signal Sig3 in which the alternate noise is effectively reduced, similarly to the foregoing embodiment and modification examples thereof.

### Modification Example G

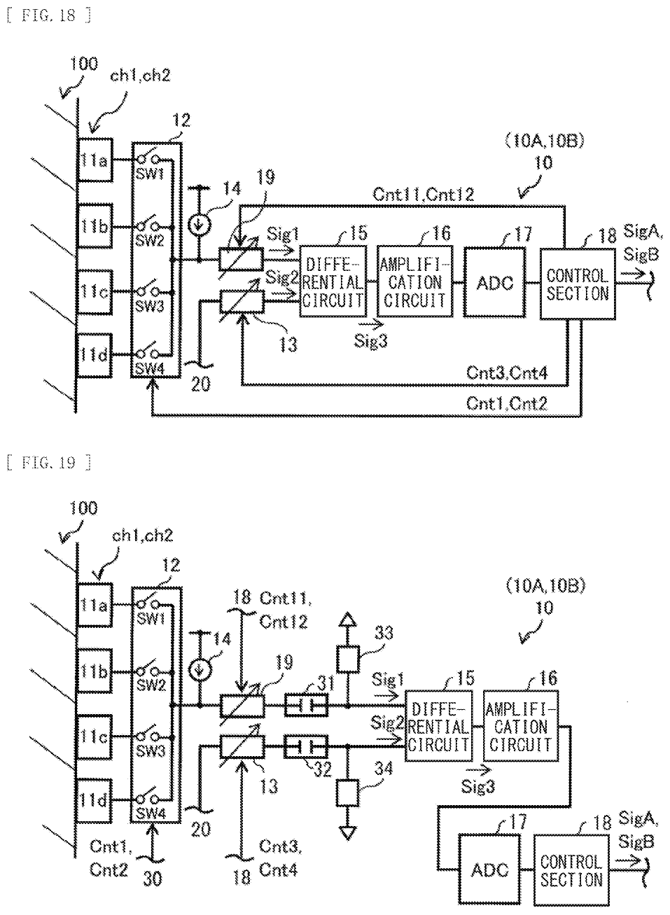

FIG. 17 illustrates a modification example of the circuit configuration of the measurement electrode module 10 and the reference electrode module 20 in the biological information measurement apparatus 1 according to any of the foregoing embodiment and modification examples thereof. In the present modification example, the communication unit 50 is omitted. In such a case, for example, the control unit 30 does not require a determination of a user, and may automatically set appropriate set values 41 and 42 of the switch elements 12 and 22 and an appropriate set value 43 of the variable resistance element 13. In addition, the control unit 30 may store the biological signals SigA and SigB obtained from the measurement electrode modules 10 (10A and 10B), for example, in the storage unit 40, without transmitting the biological signals SigA and SigB to the terminal apparatus 2 via the communication unit 50. That is, in this case, the storage unit 40 stores the biological signals SigA and SigB. Also in such a case, it is possible to obtain the biological signal Sig3 in which the alternate noise is effectively reduced, similarly to the foregoing embodiment and modification examples thereof.

### Modification Example H

In the foregoing embodiment and modification examples thereof, for example, as illustrated in FIGS. 18, 19, 20, 21, and 22, a variable resistance element 19 may be provided for a wiring line coupled to the input end on side of the measurement electrode module 10, of both the input ends of the differential circuit 15. In the variable resistance element 19, ON-OFF control is performed in accordance with a control signal Cnt11 from the control section 18 of the measurement electrode module 10A or a control signal Cnt12 from the control section 18 of the measurement electrode module 10B. A method for deriving a set value of the variable resistance element 19 is similar to the method for deriving the set value of the variable resistance element 13 in the foregoing embodiment and modification examples thereof. Thus, also in the present modification example, it is possible to obtain the biological signal Sig3 in which the alternate noise is effectively reduced, similarly to the foregoing embodiment and modification examples thereof.

### Modification Example I

In the above-described Modification Example H, for example, the variable resistance element 19 is omitted, and variable resistance elements 12A may be provided one by one for the switches SW1, SW2, SW3, and SW4 in the switch element 12 of the measurement electrode module 10, as illustrated in FIG. 23. At this time, in each of the variable resistance elements 12A, ON-OFF control is performed in accordance with a control signal Cnt13 from the control section 18. A method for deriving a set value of each of the variable resistance elements 12A is similar to the method for deriving the set value of the variable resistance element 13 in the foregoing embodiment and modification examples thereof. Thus, also in the present modification example, it is possible to obtain the biological signal Sig3 in which the alternate noise is effectively removed, similarly to the foregoing embodiment and modification examples thereof.

### Modification Example J

In the foregoing embodiment and modification examples thereof, the number of the measurement electrode modules 10 may be one or three or more. In addition, in the foregoing embodiment and modification examples thereof, the number of the reference electrode modules 20 may be two or more.

In addition, for example, the present disclosure may have the following configurations.

(1)
A biological information measurement apparatus including:
one or a plurality of measurement channels to be brought into contact with a biological body;
a reference channel to be brought into contact with the biological body;
a differential circuit that generates a biological signal corresponding to a difference between a measurement signal obtained from the measurement channel and a reference signal obtained from the reference channel; and
a switch mechanism that switches contact impedance between the biological body and each of the measurement channel and the reference channel.

(2)
The biological information measurement apparatus according to (1), in which
the measurement channel includes one or a plurality of measurement electrodes,
the reference channel includes one or a plurality of reference electrodes, and
the switch mechanism further includes
   a first switch element that selects at least one of the one or the plurality of measurement electrodes,
   a second switch element that selects at least one of the one or the plurality of reference electrodes, and
   a control section that switches the contact impedance by controlling the first switch element and the second switch element.

(3)
The biological information measurement apparatus according to (2), in which
the switch mechanism further includes a variable resistance element between the one or the plurality of reference electrodes and the differential circuit, and
the control section adjusts an impedance difference between input terminals of the differential circuit by controlling the variable resistance element.

(4)
The biological information measurement apparatus according to any one of (1) to (3), further including a storage unit that stores a first set value of the first switch element and the second switch element and a second set value of the variable resistance element, in which
the control section performs a control to switch the contact impedance and the impedance difference in an impedance measurement mode, and causes the storage unit to store the first set value and the second set value obtained thereby.

(5)
The biological information measurement apparatus according to any one of (1) to (4), in which the control section controls, in a bioelectricity measurement mode, the first switch element, the second switch element, and the variable resistance element on a basis of the first set value and the second set value obtained in the impedance measurement mode.

(6)
The biological information measurement apparatus according to any one of (1) to (5), further including an AC current source that supplies an AC current to the one or the plurality of measurement channels.

(7)
The biological information measurement apparatus according to (6), further including, between the AC current source and the differential circuit, an AC coupling circuit that performs AC measurement.

(8)
The biological information measurement apparatus according to (7), further including a third switch element coupled in parallel to the AC coupling circuit.

(9)
The biological information measurement apparatus according to (6), further including a fourth switch element that connects and disconnects the AC current source and the one or the plurality of measurement channels to and from each other.

(10)
The biological information measurement apparatus according to any one of (1) to (9), further including a transmission unit that transmits the biological signal to an external apparatus.

(11)
The biological information measurement apparatus according to any one of (1) to (9), further including the storage unit that stores the biological signal.

According to the biological information measurement apparatus of an embodiment of the present disclosure, the contact impedance is able to be adjusted depending on the contact status of the channels, thus making it possible to effectively reduce the alternate noise included in biological information even under a circumstance where the contact status of the channels may be changed. It is to be noted that the effects of the present disclosure are not necessarily limited to the effects described here, and may be any of the effects described herein.

This application claims the benefit of Japanese Priority Patent Application JP2019-006749 filed with the Japan Patent Office on Jan. 18, 2019, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

1. A biological information measurement apparatus comprising:
one or a plurality of measurement channels to be brought into contact with a biological body;
a reference channel to be brought into contact with the biological body;
a differential circuit that generates a biological signal corresponding to a difference between a measurement signal obtained from the measurement channel and a reference signal obtained from the reference channel; and
a switch mechanism that switches contact impedance between the biological body and each of the measurement channel and the reference channel.

2. The biological information measurement apparatus according to claim 1, wherein
the measurement channel includes one or a plurality of measurement electrodes,
the reference channel includes one or a plurality of reference electrodes, and
the switch mechanism further includes
a first switch element that selects at least one of the one or the plurality of measurement electrodes,
a second switch element that selects at least one of the one or the plurality of reference electrodes, and
a control section that switches the contact impedance by controlling the first switch element and the second switch element.

3. The biological information measurement apparatus according to claim 2, wherein
the switch mechanism further includes a variable resistance element between the one or the plurality of reference electrodes and the differential circuit, and
the control section adjusts an impedance difference between input terminals of the differential circuit by controlling the variable resistance element.

4. The biological information measurement apparatus according to claim 3, further comprising a storage unit that stores a first set value of the first switch element and the second switch element and a second set value of the variable resistance element, wherein
the control section performs a control to switch the contact impedance and the impedance difference in an impedance measurement mode, and causes the storage unit to store the first set value and the second set value obtained thereby.

5. The biological information measurement apparatus according to claim 4, wherein the control section controls, in a bioelectricity measurement mode, the first switch element, the second switch element, and the variable resistance element on a basis of the first set value and the second set value obtained in the impedance measurement mode.

6. The biological information measurement apparatus according to claim 1, further comprising an AC current source that supplies an AC current to the one or the plurality of measurement channels.

7. The biological information measurement apparatus according to claim 6, further comprising, between the AC current source and the differential circuit, an AC coupling circuit that performs AC measurement.

8. The biological information measurement apparatus according to claim 7, further comprising a third switch element coupled in parallel to the AC coupling circuit.

9. The biological information measurement apparatus according to claim 6, further comprising a fourth switch element that connects and disconnects the AC current source and the one or the plurality of measurement channels to and from each other.

10. The biological information measurement apparatus according to claim 1, further comprising a transmission unit that transmits the biological signal to an external apparatus.

11. The biological information measurement apparatus according to claim 1, further comprising a storage unit that stores the biological signal.
